(19) United States
(12) Patent Application Publication
(10) Pub. No.: US 2018/0153824 A1
(43) Pub. Date: Jun. 7, 2018

(54) TREATMENT OF ACUTE COMPLICATIONS OF SICKLE CELL DISEASE

(71) Applicants: NuvOx Pharma LLC, Tucson, AZ (US); University of Pittsburgh - Of the Commonwealth System of Higher Education, Pittsburgh, PA (US)

(72) Inventors: Evan C. Unger, Tucson, AZ (US); Solomon F. Ofori-Acquah, Pittsburgh, PA (US); David B. Wilson, Tucson, AZ (US)

(21) Appl. No.: 15/575,346

(22) PCT Filed: May 27, 2016
(86) PCT No.: PCT/US2016/034696

§ 371 Date: Nov. 18, 2017

Related U.S. Application Data
(60) Provisional application No. 62/167,186, filed on May 27, 2015.

(51) Int. Cl. A61K 31/02; A61P 7/00; A61K 9/107; A61K 47/24; A61P 11/00

(52) U.S. Cl. CPC A61K 31/02; A61P 7/00; A61P 11/00; A61K 47/24; A61K 9/1075

(57) ABSTRACT

The invention provides pharmaceutical compositions and dosage forms of fluorocarbon nanoemulsions that are useful for treating sickle cell disease and related diseases and conditions, as well as methods of preparation and use thereof.

### PRIORITY CLAIMS AND RELATED PATENT APPLICATIONS

This application claims the benefit of priority from U.S. Provisional Application Ser. No. 62/167,186, filed on May 27, 2015, the entire content of which is incorporated herein by reference in its entirety.

### STATEMENT OF FEDERALLY SPONSORED RESEARCH

This invention was made with Government support under HL117721 awarded by the National Institute of Health. The Government has certain rights in the invention.

### TECHNICAL FIELDS OF THE INVENTION

This invention relates to pharmaceutical compositions and methods of their preparation and therapeutic use. More particularly, the invention relates to pharmaceutical compositions and dosage forms of fluorocarbon nanoemulsions that are useful for treating sickle cell disease and related diseases and conditions, as well as methods of preparation and use thereof.

### BACKGROUND OF THE INVENTION

Sickle Cell Disease (SCD), also known as sickle cell anemia, is a group of genetically passed down blood disorders. Globally, over 3 million people are believed to have sickle-cell disease while an additional 40 million or more have sickle-cell trait. The patient population in the United States is approximately 100,000. SCD is characterized by the abnormality in the oxygen-carrying protein hemoglobin found in red blood cells. Acute chest syndrome (ACS) is the second major cause of hospital admissions in SCD patients and the number one cause of death. Although fat emboli, pneumonia, and pulmonary infarction are associated with ACS, the mechanisms that cause the lung injury in ACS have not been fully defined. Nonetheless, there is resultant hypoxemia necessitating mechanical ventilation in roughly 13% of cases and death occurs in 3% of cases.

Sickle red blood cells can cause vaso-occlusive crises by creating plugs in the vasculature. This may be an important mechanism in ACS due to pulmonary infarction. Patients with SCD are subject to strokes, renal damage, eye damage, lung damage, bone infarcts, splenic infarction, and hepatic damage. Vaso-occlusive disease in sickle cell crisis is an important factor in all of these conditions.

Available interventions are not optimal in providing hastened recovery of lung function and diminishing pain associated with vaso-occlusive crisis or sickle cell crisis (SCC). On average a SCD patient with ACS spends more than 10 days in the hospital. Delayed restoration of critical oxygenation to tissues can lead to end-organ damage.

Thus, an urgent need remains for a safe and reliable therapy that can be deployed early to SCD patients in crisis and help restore critical oxygen supply to organs affected by vaso-occlusive disease to reduce ischemic tissue damage, improve treatment outcome and lower healthcare costs.

### SUMMARY OF THE INVENTION

The invention is based in part on the unexpected discovery of pharmaceutical compositions of certain fluorocarbons that exhibit exceptional therapeutic properties and can be safely and reliably used for treating SCD patients in SCC. The unique pharmaceutical compositions and methods of use disclosed herein enable early intervention and timely restoration of critical oxygen supply to affected organs, thus leading to reduced ischemic tissue damage and improved treatment outcome.

In one aspect, the invention generally relates to a method for treating sickle cell disease, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

In another aspect, the invention generally relates to a method for treating a lung condition, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

In yet another aspect, the invention generally relates to a pharmaceutical composition comprising a dosage of a fluorocarbon having a boiling point between about −4° C. and about +100° C. therapeutically effective to treat sickle cell disease, or a related disease or disorder thereof, in a mammal, including a human, and a pharmaceutically acceptable carrier or excipient.

In yet another aspect, the invention generally relates to a unit dosage form of a pharmaceutical composition in the form of a nanoemulsion comprising a therapeutically effective dosage of a fluorocarbon having a boiling point between about −4° C. and about +100° C., and a pharmaceutically acceptable carrier or excipient.

### BRIEF DESCRIPTION OF THE DRAWINGS

The invention will be better understood from a reading of the following detailed description taken in conjunction with the drawings in which like reference designators are used to designate like elements, and in which:

FIG. 1A graphically illustrates mean oxygen saturation (SpO₂) values for SS mice (n=3 in each group) administered NVX-108 or saline (vehicle), followed by a challenge with i.v. hemin to induce ACS. After initial hypoxemia in both groups of animals, SpO₂ recovered in the NVX-108-treated but not in the saline-treated SS mice.

FIG. 1B graphically illustrates that transgenic SCD mice pretreated with NVX-108 had a 50% survival while all the saline pretreated animals succumbed to hemin-induced ACS. P<0.001.

FIG. 2 graphically illustrates oxygen saturation of transgenic SCD mice with induced ACS followed by the administration of either NVX-108 or saline.

FIG. 3A graphically illustrates an edema assessment by wet/dry lung weight ratio that indicated survival of NVX-108 treated SS mice was not due to fluid clearance.

FIG. 3B shows low-power image of stained lung tissue sections showing a remarkable degree of lack of vascular congestion in the lungs of SS mice with ACS treated with NVX-108.

### DEFINITIONS

Unless defined otherwise, all technical and scientific terms used herein have the same meaning as commonly understood by one of ordinary skill in the art to which this invention belongs.

As used herein, “NVX-108” refers to a dodecafluoropentane (DDFP) nanoemulsion (DDFPe) stabilized by fluorosurfactant, PEG-Telomer B and suspended in 30% sucrose.

As used herein, the term “nanoemulsion” refers to a suspension or emulsion of nanodroplets in aqueous media. Nandroplet refers to submicron droplets comprising a liquid fluorocarbon ranging from 4 carbond to 8 carbons in length (preferably 5 carbons, preferably dodecafluoropentane.

As used herein, “administration” of a disclosed compound or composition encompasses the delivery to a subject of a pharmaceutical composition using any suitable formulation or route of administration, as discussed herein.

As used herein, the terms “effective amount” or “therapeutically effective amount” refer to that amount of a compound or pharmaceutical composition described herein that is sufficient to effect the intended benefit including, but not limited to, disease treatment, as illustrated herein. The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

As used herein, the “high dosage” is meant at least 5% (e.g., at least 10%, 20%, 50%, 100%, 200%, or even 300%) more than the highest standard recommended dosage of a particular compound for treatment of any human disease or condition.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The invention provides compositions of certain fluorocarbons that exhibit exceptional therapeutic properties and can be safely and reliably used for treating SCD patients in vaso-occlusive crises. The unique pharmaceutical compositions and methods of use disclosed herein enable early intervention and timely restoration of critical oxygen supply to affected organs, thus leading to reduced ischemic tissue damage and improved treatment outcome.

Oxygenated perflubron emulsion (perfluorooctyl bromide, trade name Imagent) has been tested in a preclinical model of SCD. Perflubron-based therapies require high doses and their development has been terminated due to limited efficacy and adverse events. Perfluoro-tert-butylcyclohexane (trade name Oxycyte) was tested in a model of S. pneumonia in SCD mice. HbSS mice treated with 3 mL/Kg (1.8 g/Kg) oxygenated perfluoro(tert-butylcyclohexane) emulsion (PFCE-O₂) had significantly better survival than HbSS littermates treated with PFCE-air, Oxygenated Phosphate buffered saline (PBS-O₂,) or Aerated Phosphate Buffered Saline (PBS-air). The dose of Oxycyte was 3 mL/Kg (60% weight/volume) in this study. As shown below, dodecafluoropentane emulsion (DDFPe) was active at a dose of 0.6 mL/Kg (2% weight/volume=12 mg/kg). In other words, the dose of DDFPe was active at 1/150^{th} the dose of Oxycyte. Development of Oxycyte was terminated apparently due to poor safety and limited efficacy. None of these fluorocarbon-based compositions have entered into clinical trials to treat patients with SCD crisis. The prior materials failed due to high doses, limited efficacy and adverse side effects. The much lower dose and greater efficacy of the fluorocarbons of this invention yield a favorable safety factor and therapeutic index affording multi-dose administration. Note that none of the prior agents were capable of multi-dose administration to treat sickle cell crisis.

In one aspect, the invention generally relates to a method for treating sickle cell disease, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

In another aspect, the invention generally relates to a method for treating a lung condition, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

In yet another aspect, the invention generally relates to a pharmaceutical composition comprising a dosage of a fluorocarbon having a boiling point between about −4° C. and about +100° C. therapeutically effective to treat sickle cell disease, or a related disease or disorder thereof, in a mammal, including a human, and a pharmaceutically acceptable carrier or excipient.

In yet another aspect, the invention generally relates to a unit dosage form of a pharmaceutical composition in the form of a nanoemulsion comprising a therapeutically effective dosage of a fluorocarbon having a boiling point between about −4° C. and about +100° C., and a pharmaceutically acceptable carrier or excipient.

In certain embodiments, the fluorocarbon is preferably stabilized in the form of a nanoemulsion. In certain preferred embodiments, the pharmaceutical composition is a nanoemulsion, e.g., a homogenized nanoemulsion. In certain embodiments, the nanoemulsions comprise particles having a maximum dimension less than about 1 μm in size. In certain embodiments, the nanoemulsions comprise particles having a mean size of about 500 nm in size. In certain embodiments, the nanoemulsions comprise particles having a mean size of about 250 nm. In certain embodiments, the nanoemulsions comprise particles having a mean size of about 200 nm.

In certain embodiments, the boiling point of the fluorocarbon used is preferably between about 28° C. and about 60° C. In certain embodiments, the fluorocarbon used preferably has between 4 and 8 linear and/or branched carbon atoms with from about 10 to about 18 fluorine atoms.

Fluorocarbons useful in the invention include perfluorobutane, perfluoropentane, perfluorohexane, perfluoroheptane and perfluorooctane, or a mixture of two of more thereof. In certain embodiments, the pharmaceutical composition utilizes perfluorohexane and/or perfluoropentane. In certain embodiments, the pharmaceutical composition utilizes perfluoropentane. Perfluoropentane may comprise isomers of dodecafluoro-n-pentane and dodecafluoro-iso-pentane.

In one example, based on an initial study as disclosed herein using an emulsion of DDFP has been used to treat ACS at 1.0 mL/Kg dose (2% w/vol DDFP) with an effective dose of 20 mg/Kg. Such a dose is 1/90^{th} of the dose of the fluorocarbon of Oxycyte.

The fluorocarbon accounts for a weight percent in the nanoemulsion from about 1% to about 50%. In certain embodiments, the fluorocarbon accounts for a weight percent in the nanoemulsion from about 1% to about 10%.

In certain embodiments, the nanoemulsion has from about 0.5 to about 20% w/vol of fluorocarbon. In certain embodiments, the nanoemulsion has between about 1 and about 10% w/vol fluorocarbon. In certain embodiments, the nanoemulsion has between about 1 and about 5% w/vol fluorocarbon. In certain embodiments, the nanoemulsion has between about 5 and about 10% w/vol fluorocarbon. In certain embodiments, the nanoemulsion has between about 1 and about 3% w/vol fluorocarbon. In certain embodiments, the nanoemulsion has between about 3 and about 5% w/vol fluorocarbon.

In certain embodiments, the fluorocarbon is stabilized by one or more surfactants. For example, surfactants may be one or more fluorosurfactants such as PEG-Telomer-B, CAPSTONE, diacylglycerophospholipids, cholesterol, and/or other surfactants known in the art. In certain embodiments, the surfactant(s) utilized comprise one or more fluorosurfactants and one or more phospholipids. In certain embodiments, the surfactant(s) is incorporated into the nanoemulsion in amounts ranging from about 0.1% weight volume to about 10% weight volume. In certain embodiments, the surfactant(s) is incorporated into the nanoemulsion in amounts ranging from about 0.2% w/vol to about 2% w/vol.

In certain embodiments, the pharmaceutical composition comprises one or more phospholipids having carbon chains ranging from about 12 carbons to about 18 carbons in length.

In certain embodiments, the phospholipids accounts for a weight percent in the pharmaceutical composition from about 0.10% to about 7.5%.

Any suitable therapeutically effective dosage may be employed, for example, a dosage that ranges from about 2.0% to about 4.0%. In certain embodiments, the therapeutically effective dosage ranges from about 4.0% to about 6.0%.

In certain embodiments, a dose of about 0.5 mg/Kg to about 5 mg/Kg is administered. In certain embodiments, a dose of about 1.0 mg/Kg to about 3.5 mg/Kg is administered. In certain embodiments, a dose of about 1.5 mg/Kg to about 2.5 mg/Kg is administered. In certain embodiments, a dose of about 2.0 mg/Kg is administered.

In certain embodiments, a dose is repeated from about 60 min. to about 120 min. (e.g., about 60 min. to about 90 min., about 90 min. to about 120 min., about 60 min., about 90 min., about 120 min.) apart for 2, 3, 4, 5 or 6 times. In certain embodiments, the dose is repeated from about 90 min. to about 120 min. apart for 2 times. In certain embodiments, the dose is repeated from about 90 min. to about 120 min. apart for 3 times. In certain embodiments, the dose is repeated from about 90 min. to about 120 min. apart for 4 times. In certain embodiments, the dose is repeated from about 90 min. to about 120 min. apart for 5 times. In certain embodiments, the dose is repeated from about 90 min. to about 120 min. apart for 6 times.

Any suitable therapeutically effective dosage unit dosage form may be employed, for example, comprising about 2% to about 4% of the fluorocarbon. In certain embodiments, the unit dosage form comprises about 4% to about 6% of the fluorocarbon. In certain embodiments, the unit dosage form comprises from about 7 mg to about 140 mg (e.g., about 7 mg to about 100 mg, about 7 mg to about 70 mg, about 7 mg to about 35 mg, about 35 mg to about 140 mg, about 70 mg to about 140 mg, about 35 mg to about 70 mg) of fluorocarbon.

In certain embodiments, the fluorocarbon nanoemulsion is administered IV to treat SCC. In certain embodiments, the dose is between about 1 mg/Kg to about 100 mg/Kg fluorocarbon. In the case of the 2% w/vol DDFPe, the dose is from about 0.01 mL/Kg to about 1.0 mL/Kg. In certain embodiments, the dose is from about 0.05 mL/Kg to about 0.5 mL/Kg fluorocarbon to treat a human patient. In certain embodiments, the dose is from about 0.05 mL/Kg to about 0.1 mL/Kg fluorocarbon to treat a human patient. In certain embodiments, the dose is from about 0.1 mL/Kg to about 0.3 mL/Kg fluorocarbon to treat a human patient. In certain embodiments, the dose is from about 0.3 mL/Kg to about 0.5 mL/Kg fluorocarbon to treat a human patient.

In certain embodiments, the fluorocarbon may be administered as an IV bolus. In certain embodiments, the fluorocarbon may be administered by sustained IV infusion. The concentration of fluorocarbon in the nanoemulsion can be increased, for example, up to about 60% weight/vol if desired, to minimize the volume injected.

Hemolysis is a common condition present in SCC. The oxidized byproduct of hemolysis, hemin, exacerbates the symptoms associated with SCC in animal models in a process involving TLR4 signaling. In certain embodiments, the fluorocarbon nanoemulsion may be co-administered with anti-inflammatory agents to ameliorate the sequelae of sickle crisis.

The fluorocarbon nanoemulsions of the invention may be co-administered with one or more other suitable agents, or one or more such other agents may be incorporated into the fluorocarbon nanoemulsion. For example, a TLR4 inhibitor may be co-administered or incorporated into the fluorocarbon nanoemulsion of the invention. Examples of such agents include TAK-242 (with the trade name Resatorvid), a small-molecule—specific inhibitor of Toll-like receptor (TLR) 4 signaling, which has been shown to inhibit the production of NO and pro-inflammatory cytokines. TAK-242 acts by blocking the signaling mediated by the intracellular domain of TLR4, but not the extracellular domain. TAK-242 potently suppresses both ligand-dependent and -independent signaling of TLR4.

Another example of a TLR4 inhibitor is C34 (a.k.a. TLR4-IN-C34, with the formula C₁₇H₂₇NO₉), which can be used co-administered or incorporated with the fluorocarbon of the invention. Other TRL4 inhibitors that may be used in the invention include amitriptyline, cyclobenzaprine, ibudilast, imipramine, ketotifen, mianserin, naloxone, naltrexone, (+)-naltrexone, propentofylline, LPS-RS and (+)-naloxone.

OxPAPC inhibits TLR2 and LR4. It is generated by the oxidation of 1-palmitoyl-2-arachidonyl-sn-glycero-3-phosphorylcholine (PAPC), which results in a mixture of oxidized phospholipids containing either fragmented or full length oxygenated sn-2 residues. OxPAPC has been shown to inhibit the signaling induced by bacterial lipopeptide and lipopolysaccharide (LPS). OxPAPC acts by competing with CD14, LBP and MD2, the accessory proteins that interact with bacterial lipids, thus blocking the signaling of TLR2 and TLR4. PAPC, 1-palmitoyl-2-arachidonyl-sn-glycero-3-phosphorylcholine, can be incorporated into the nanoemulsion stabilizing the fluorocarbon. In certain embodiments, OxPAPC can be co-administered with the FC to improve treatment of SCC.

Hemopexin, also known as the beta-1B-glycoprotein, is a protein that scavenges and binds heme more tightly than any other protein. In certain embodiments, Hemopexin may be co-administered with the fluorocarbon nanoemulsion of the invention to improve treatment of SCC.

Antioxidants may also be used in the invention to improve the activity of the fluorocarbon. Examples of useful antioxidants include n-acetylcysteine, ascorbic acid, and a-tocopherol. In certain embodiments, n-acetylcysteine can be administered at 150 mg/Kg for 30 min then 20 mg/Kg/h plus bolus doses of 1 g ascorbic acid and 400 mg α-tocopherol.

The following examples are presented to further illustrate to persons skilled in the art how to make and use the invention. These examples are not intended as a limitation, however, upon the scope of the invention.

### EXAMPLES

### Example 1

A 30% sucrose solution was prepared by dissolving an appropriate amount of USP grade sucrose in water for injection at room temperature followed by a mixture of disodium hydrogen phosphate and sodium dihydrogen phosphate to buffer the system at a pH of 7.0. In a second vessel a suspension of DDFP (dodecafluoropentane) in PEG-Telomer B in the ratio of DDFP:PEG-Telomer B:5:1 (w:w), was prepared as follows: PEG-Telomer B was dispersed in water for injection by stirring in a jacketed vessel cooled to 4° C. Pre-cooled (4° C.) DDFP was added to the stirred PEG-Telomer B and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water. The resulting solution was stirred for up to 20 minutes, and then transferred through the homogenizer under low pressure to a third vessel. This solution was then transferred through a 0.2 micron filter into a fourth vessel. The product was dispensed to vials, which were capped and crimped. These operations were carried out at <8° C. in cold jacketed vessels due to the volatility of the active ingredient (DDFP). Compensation for losses during processing was accounted for by the use of an overage of the active component. Product fill volume was also tightly controlled to produce vials to meet release and shelf-life specifications. The resulting product comprised 2% w/vol DDPE. Particle sizing by Nycomp showed mean particle size of about 250 nm.

### Example 2

A 5% sucrose solution was prepared by dissolving an appropriate amount of USP grade sucrose in water for injection at room temperature followed by a mixture of disodium hydrogen phosphate and sodium dihydrogen phosphate to buffer the system at a pH of 7.0. In a second vessel a suspension of DDFP (dodecafluoropentane) in PEG-Telomer B in the ratio of DDFP:PEG-Telomer B:5:1 (w:w), was prepared as follows: PEG-Telomer B was dispersed in water for injection by stirring in a jacketed vessel cooled to 4° C. Pre-cooled (4° C.) DDFP was added to the stirred PEG-Telomer B and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water. The resulting solution was stirred for up to 20 minutes, and then transferred through the homogenizer under low pressure to a third vessel. This solution was then transferred through a 0.2 micron filter into a fourth vessel. The product was dispensed to vials, which were capped and crimped. These operations were carried out at <8° C. in cold jacketed vessels due to the volatility of the active ingredient (DDFP). Compensation for losses during processing was accounted for by the use of an overage of the active component. Product fill volume was also tightly controlled to produce vials to meet release and shelf-life specifications. The resulting product comprised 2% w/vol DDPE. Particle sizing by Nicomp showed mean particle size of about 250 nm.

### Example 3

A suspension of a mixture of phospholipids with the following composition, Dipalmitoylphosphatidylcholine (DPPC) and Phosphatidylethanolamine-PEG 5k in a mole ratio of 92 mole percent DPPC and 8 mole percent DPPE-PEG was prepared by warming them in a mixture of propylene glycol (15 v %), Glycerol (5 v %) and 5 mM sodium phosphate in water buffered 0.9% normal saline (85 v %), to above the phase transition temperature of the all the lipids.

After the lipids were dispersed the suspension was stirred in a jacketed vessel and cooled to 4° C. Pre-cooled (4° C.) DDFP was added to the stirred phospholipid suspension at weight ratio of 5 to 1, and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water.

The resulting solution was stirred for up to 20 minutes, and then transferred through the homogenizer under low pressure to a third vessel. This solution was then transferred through a 0.2 micron filter into a fourth vessel. The product was dispensed to vials, which were capped and crimped. These operations were carried out at <8° C. in cold jacketed vessels due to the volatility of the active ingredient dodecafluoropentane (“DDFP”). Compensation for losses during processing is accounted for by the use of an overage of the active component. Product fill volume was also tightly controlled to produce vials to meet release and shelf-life specifications.

### Example 4

A suspension of a mixture of phospholipids with the following composition, Dipalmitoylphosphatidylcholine (DPPC) and Phosphatidylethanolamine-PEG 5k in a mole ratio of 92 mole % DPPC and 8 mole % DPPE-PEG was prepared by warming them in a mixture of propylene glycol (15 v %) , Glycerol (5 v %) and 5 mM sodium phosphate in water buffered 0.9% normal saline (85 v %), to above the phase transition temperature of the all the lipids.

Pre-cooled (4° C.) perfluorohexane was added to the stirred phospholipid suspension at weight ratio of 7 to 1, and allowed to stir until a uniformly milky suspension was achieved. This suspension was homogenized under high pressure in an Avestin model C50 homogenizer for up to 18 minutes keeping the temperature below 7° C.). The emulsion was transferred via the homogenizer under low pressure to a vessel containing 30% sucrose solution in water; the resulting solution was stirred for up to 20 minutes, and then transferred through the homogenizer under low pressure to a third vessel. This solution was then transferred through a 0.2 micron filter into a fourth vessel. The product was dispensed to vials, which were capped and crimped. These operations were carried out at <8° C. in cold jacketed vessels due to the volatility of the active ingredient (perfluorohexane). Compensation for losses during processing was accounted for by the use of an overage of the active component. Product fill volume was also tightly controlled to produce vials to meet release and shelf-life specifications.

### Example 5

A suspension of a mixture of phospholipids with the following composition, Dipalmitoylphosphatidylcholine (DPPC) and Phosphatidylethanolamine-PEG 5k in a mole ratio of 92 mole % DPPC and 8 mole % DPPE-PEG at a total concentration of 3mg/mL was prepared by warming them in a mixture of propylene glycol (15 v %) , Glycerol (5 v %) and 5 mM sodium phosphate in water buffered 0.9% normal saline (85 v %), to above the phase transition temperature of the all the lipids.

Pre-cooled (4° C.) perfluoroheptane was added to the stirred phospholipid suspension at weight ratio of 7 to 1, and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water; the resulting solution was stirred for up to 20 minutes, and then transferred through the homogenizer under low pressure to a second vessel. This solution was then transferred through a 0.2 micron filter into a third vessel. The product was dispensed to vials, which were capped and crimped. These operations were carried out at <8° C. in cold jacketed vessels due to the volatility of the active ingredient perfluoroheptane. Compensation for losses during processing was accounted for by the use of an overage of the active component. Product fill volume was also tightly controlled to produce vials to meet release and shelf-life specifications.

### Example 6

A suspension of a mixture of phospholipids with the following composition, Dipalmitoylphosphatidylcholine (DPPC) and Phosphatidylethanolamine-PEG 5k in a mole ratio of 92 mole % DPPC and 8 mole % DPPE-PEG was prepared at total concentration of 3 mg/mL by warming them in a mixture of propylene glycol (15 v %), Glycerol (5 v %) and 5 mM sodium phosphate in water buffered 0.9% normal saline (85 v %), to above the phase transition temperature of the all the lipids.

Pre-cooled (4° C.) perfluorooctane was added to the stirred phospholipid suspension at weight ratio of 7 to 1, and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water; the resulting solution is stirred for up to 20 minutes, and then transferred through the homogenizer under low pressure to a third vessel. This solution was then transferred through a 0.2 micron filter into a fourth vessel. The product was dispensed to vials, which were capped and crimped. These operations are carried out at <8° C. in cold jacketed vessels due to the volatility of the active ingredient (perfluorooctane). Compensation for losses during processing were accounted for by the use of an overage of the active component. Product fill volume was also tightly controlled to produce vials to meet release and shelf-life specifications.

### Example 7

The materials of Example 1 are used with a hand-held homogenizer, except that sucrose level is lowered to 10% by weight, along with buffered saline is used as the suspending medium. The process yields an emulsion that is similar to that obtained from Example 1, except that the nanoparticles were noted to settle to the bottom of the sealed vials more quickly than material in Example #1 that contained sucrose in the suspending media. The nanoparticles, however, could be easily resuspended by agitating the vials by hand or by vortexing.

### Example 8

A suspension of a mixture of phospholipids with the following composition, Dipalmitoylphosphatidylcholine (DPPC) and Phosphatidylethanolamine-PEG 5k in a mole ratio of 92 mole % DPPC and 8 mole % DPPE-PEG was prepared at total concentration of 3 mg/mL by warming them in a mixture of propylene glycol (15 v %) , Glycerol (5 v %) and 5 mM sodium phosphate in water buffered 0.9% normal saline (85 v %), to above the phase transition temperature of the all the lipids. Once the lipids have been suspended Capstone at a concentration of 3 mg/mL is added to lipid suspension until completely dispersed.

Pre-cooled (4° C.) DDFP was added to the stirred phospholipid suspension at weight ratio of 7 to 1, and allowed to stir until a uniformly milky suspension was achieved. This suspension was homogenized under high pressure in an Avestin model C50 homogenizer for up to 18 minutes keeping the temperature below 7° C. The emulsion was transferred via the homogenizer under low pressure to a vessel containing 30% sucrose solution in water; the resulting solution is stirred for up to 20 minutes, and then transferred through the homogenizer under low pressure to a third vessel. This solution was then transferred through a 0.2 micron filter into a fourth vessel. The product was dispensed to vials, which were capped and crimped. These operations are carried out at <8° C. in cold jacketed vessels due to the volatility of the active ingredient (perfluorooctane). Compensation for losses during processing were accounted for by the use of an overage of the active component. Product fill volume was also tightly controlled to produce vials to meet release and shelf-life specifications

### Example 9

Hemin is a potent inflammatory agonist, and activator of TLR4, and thus a potential Danger Associated Molecular Pattern (DAMP) molecule. Enhanced auto-oxidation of Hb S, and the low steady-state levels of haptoglobin (the plasma Hb scavenger molecule) in SCD promote the conversion of extracellular oxyHb to ferric metHb, and consequently elevation of extracellular hemin concentrations. A diversity of clinical and genetics evidence implicate hemin in the pathogenesis of ACS: a) acute hemolysis is a predictor of sudden death in ACS, b) oxidative stress (which promotes hemin release from Hb) increases during ACS, and c) SCD patients with a polymorphism that increases expression of heme oxygenase-1 (the rate-limiting hemin degradation enzyme) have lower rates of incidence of ACS.

The first preclinical murine model of ACS was recently developed based on the infusion of hemin into transgenic SCD (SS) mice. (Ghosh, Samit, et al. “Extracellular hemin crisis triggers acute chest syndrome in sickle mice.” The Journal of Clinical Investigation 123.11 (2013): 4809-4820.) In this model, the sickle (SS) but not the sickle-trait (AS) mice develop all the major clinical (acute illness), pathological (hypoxemia) and biological (pulmonary infiltration/edema) hallmarks of human ACS.

To assess the therapeutic feasibility of using DDFPe to treat ACS, NVX-108 (NuvOx Pharma LLC) was tested is effective in reducing mortality, hypoxemia and lung injury (edema, congestion) in this murine ACS model.

NVX-108 comprises a formulation having the components recited in Table 1.

TABLE 1

| | | | Concentration |
|---|---|---|---|
| Ingredient | Specification | Purpose | (mg/mL) |
| Dodecafluoropentane | Medical Grade | Active | 20 |
| Sucrose | Medical Grade | Excipient | 300 |
| PEG-Telomer B | Purified Chemical | Excipient | 3 |
| | Grade | | |
| Water for Injection | USP | Solvent | q.s. to 1 mL |
| Nitrogen | Medical Grade | Head space | q.s. |
| | | Air Flush | |
| Sodium Phosphate | USP | Buffer | 0.01M |
| Hydrochloric acid | USP | Excipient | q.s. |

First, NVX-108 was tested as a prophylactic. Adult SCD mice (12-14 weeks) breathing room air were infused with NVX-108 (1 mL/Kg bw) or saline via the lateral tail vein, challenged with 70 μmoles/Kg bw of i.v. hemin and monitored for 2 hrs. All the SS mice given saline (n=3) died within 2 hrs, while three of the six mice given DDFPe survived. In the next experiment, Applicants monitored peripheral capillary oxygen saturation (SpO₂) continuously using a mouse pulse oximeter validated previously against a blood gas analyzer.

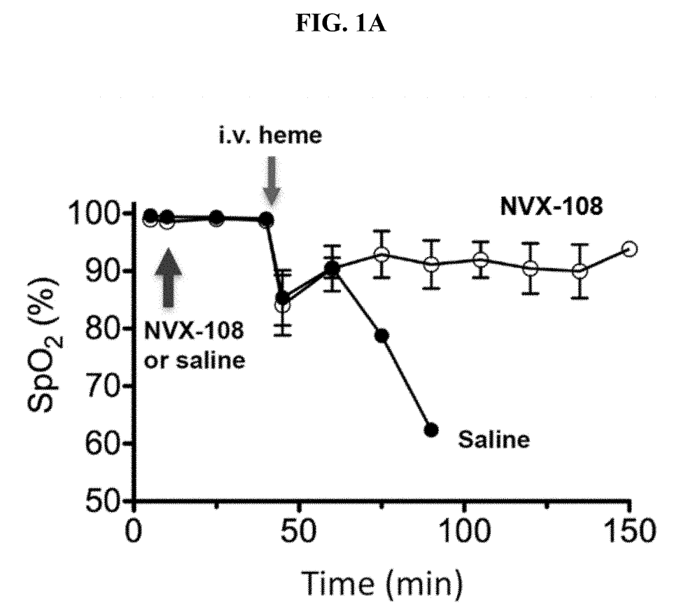

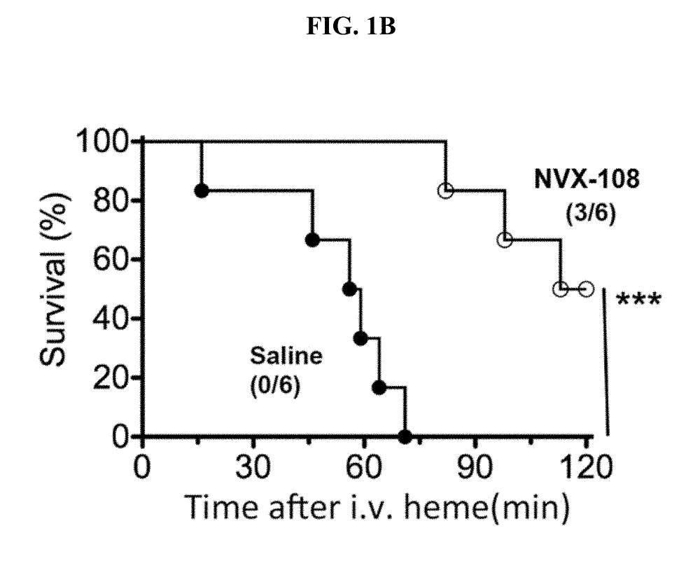

Prior to the hemin challenge, both groups of SS mice (i.e., DDFPe and saline) had comparable and stable levels of SpO₂ of ˜99% (FIG. 1A). Within 5 min of the hemin challenge, the SpO₂ declined in both (DDFPe: 84.1 ±5%, n=3; Saline: 85±4.8%, n=3). This hypoxemia was transient as values recovered to ˜90% within 15 min in both groups. (FIG. 1A.)

The re-oxygenation of blood in room air continued in the DDFPe but not in the saline-treated SS mice. One of the saline-treated animals died 20 min after the hemin challenge, and the other two died at 35 min and 50 min, all with symptoms of respiratory distress. Two DDFPe-treated animals survived the ACS with final SpO₂ values of 93% and 94% while the third mouse succumbed 35 min after the hemin challenge ironically with a higher SpO2 value of 96.6%. Thus, infusion DDFPe prophylaxis attenuated hypoxemia and reduced mortality by 50% in SS mice experiencing ACS. (FIG. 1B.)

To determine whether infusion of NVX-108 will be efficacious also as a therapeutic, Applicants administered the drug (or a saline vehicle) to SS mice after they had been challenged with i.v. hemin. Applicants previously reported, and confirmed here in preliminary studies, that SS mice develop early signs of ACS (i.e., respiratory distress) ˜5 min after being challenged with hemin, (FIG. 1A.) Thus, while SpO₂ was being recorded in mice breathing room air, they were challenged with i.v. hemin (70 μmoles/Kg bw), followed five minutes later with infusion NVX-108 (n=3) or saline (n=3).

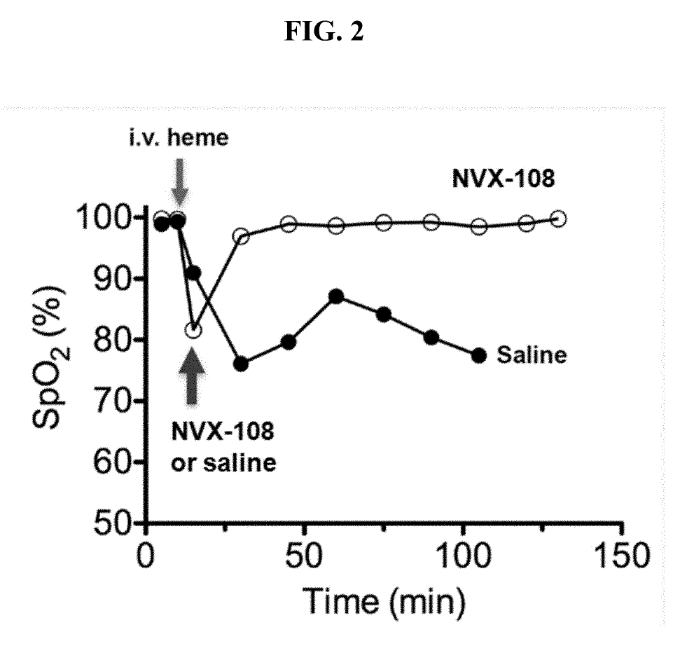

Recovery of SpO₂ was sustained in all the NVX-108 treated mice, and in agreement with Applicants' published data, the saline-treated animals developed respiratory failure and died. (FIG. 2.) All three animals treated with NVX-108 survived despite the presentation of early signs of ACS (data not shown). Immediately following death or at the end of the ACS experiment (i.e., 2 hours after the i.v. hemin), lungs from the mice were harvested for gravimetric assessment of edema formation and histological analysis for other signs of lung damage.

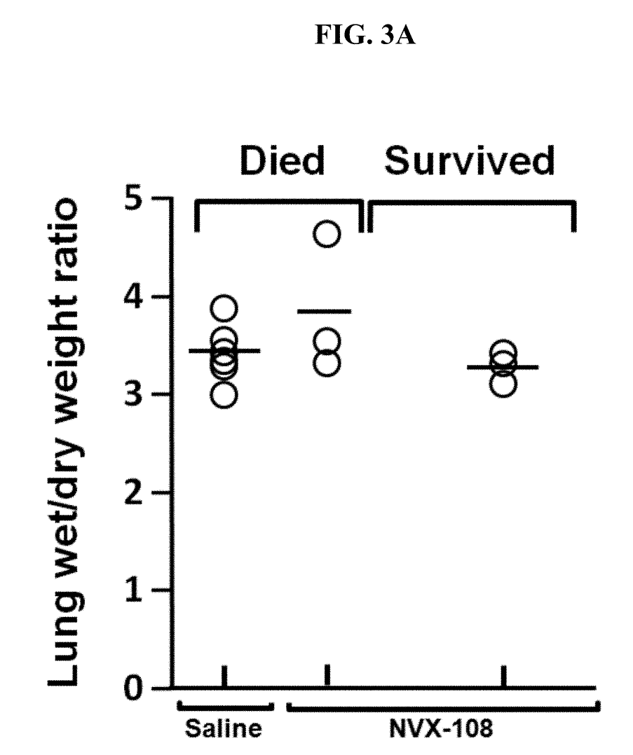

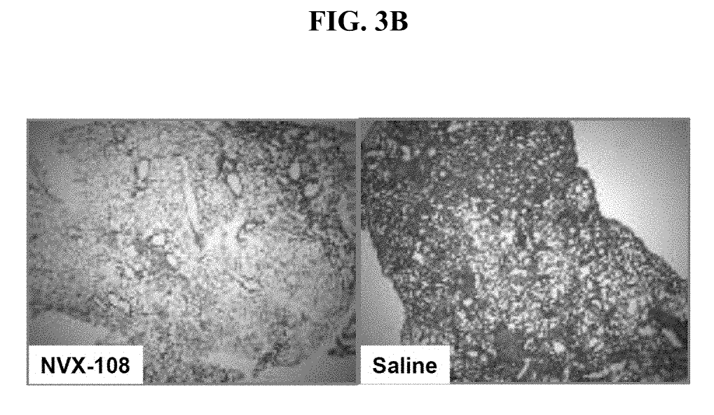

Results show comparable lung wet/dry weight ratios for SS mice that succumbed or survived the hemin induced ACS regardless of NVX-108 or saline prophylaxis or treatment. See, FIG. 3A. This result suggest that the ameliorating effects of NVX-108 in the SS mice maybe independent of fluid clearance in the lungs. However, compared to saline-treated animals that all succumbed, the lungs of SS mice that survived the ACS after being treated with NVX-108 were remarkably uncongested. (FIG. 3B). These preliminary findings suggest NVX-108 enhanced pulmonary micro-vascular blood flow, presumably due to effective re-oxygenation and the un-sickling of sickle erythrocytes.

### Example 8

A patient with SCD presents with chest pain, labored breathing and hypoxemia. A diagnosis of acute chest syndrome is made. The patient is placed on nebulized oxygen and receives an IV infusion of 0.2 mL/Kg 2% w/vol DDFPe. The chest pain resolves and the oximetry readings show resolution of hypoxia.

### Example 9

A pediatric patient with SCD presents with pain in the joints (knees and hips). A diagnosis of vaso-occlusive crisis is made. The patient receives an IV infusion of 1 mL/Kg of 10% w/vol perfluorohexane emulsion stabilized with DPPC/DPPE-PEG5,000. The patient's pain resolves. The patient is able to return home without need for hospitalization.

### Example 10

A patient presents with SCC and signs of shock. An emulsion of 4 w/vol % perfluorohexane is administered IV at a dose of 0.4 mL/Kg. The antioxidant, n-acetylcysteine is administered at 150 mg/Kg for 30 min then 20 mg/Kg/h plus bolus doses of 1 g ascorbic acid and 400 mg a-tocopherol IV. The patient recovers and has a good outcome.

### Example 11

An emulsion of DDFPe is formulated from DDFP using phospholipids enriched with 1-palmitoyl-2-arachidonyl-sn-glycero-3-phosphorylcholine (PAPC). The material is useful for treating SCC by not only delivering oxygen but also by inhibiting TLR4.

### Example 12

Acute Lung Injury (ALI) and Acute Respiratory Distress Syndrome (ARDS) are conditions where lungs fill with fluid and inflammatory cells resulting in impaired oxygen and carbon dioxide exchange. ALI and ARDS occur most commonly from pneumonia, but can also be caused by trauma, sepsis and other conditions. Influenza pneumonia is the causative agent in about a quarter of the cases of ALI and ARDS. The mortality of ARDS has improved with better supportive care but is still very high, wherein about 22% of patients die within onset of ARDS. A consensus conference recommended criteria for classification of ALI and ARDS as follows: arterial hypoxemia with PaO₂/FiO₂ (partial pressure arterial oxygen/fraction of inspired oxygen) ratio less than 300 mmHg for ALI and less than 200 mmHg to define ARDS, and for ARDS bilateral radiographic opacities.

A recent report entitled “the Berlin definition,” recommends use of three categories of ARDS, based on the degree of hypoxemia: mild (200 mmHg <PaO₂/FiO₂≤300 mmHg), moderate (100 mmHg<PaO₂/FiO₂≤200 mmHg), and severe (PaO₂/FiO₂≤100 mmHg). Regardless of the categorization, the worse the hypoxemia in ALI and ARDS the worse the mortality. To raise paO₂ in patients with severe ARDS the FiO₂ is often raised to compensate.

High FiO₂, for example, near or greater than 50%, may result in oxygen toxicity damaging the pulmonary tissue. Also to compensate for impaired elasticity in the lungs and impaired oxygen/carbon dioxide exchange the ventilator pressure is often increased, and high-pressures further damage the pulmonary tissue. Some goals in the management of ARDS, to decrease complications and damage to the lungs, are to maintain an end-inspiratory plateau pressure of the respiratory system (Plats), measured after a 1-second period of no airflow, of no more than 30 cm of water and an arterial plasma pH of 7.20 to 7.45.

DDFPe will be useful in ARDS because PaO₂ will rise at lower FiO₂, and lower positive pressures will be needed for ventilation. Use of DDFPe will decrease mortality and improve outcomes in ARDS. Lower end-inspiratory plateau pressures will be needed using the invention, e.g. <30 cm water and arterial plasma pH will be more easily maintained in the range of pH=7.20 to 7.45.

### Example 13

A 41-year old man presents with a two-day history of myalgias and fever, a productive cough, and shortness of breath. Chest radiography shows patchy bilateral infiltrates in the lungs. Diagnostic evaluation confirmed H1N1 influenza infection. Because of worsening hypoxia and difficulty breathing, the patient is intubated and mechanically ventilated. The PaO₂/FiO₂ ratio is less than 200 mm/Hg and a diagnosis of ARDS is made. The patient is administered IV boluses of DDFPe, 0.17 mL/Kg, 2% w/vol emulsion, about 90 minutes apart. After the first injection paO increases and after four doses the patient is extubated.

### Example 14

A patient with ALI has a PaO₂/FiO₂ ratio less than 300 mmHg. The patient is administered emulsified perfluorohexane, 0.1 mL/Kg of 10% w/vol emulsion. Mechanical ventilation had been considered but was deemed not necessary due to the patient's improved condition after administration of the emulsion.

### Example 15

An emulsion is prepared as in Example 6 except that the phospholipids are enriched with 10 mole % sphingosine-1-phosphate. These emulsions are used to treat patients with ARDS and there is improved resolution of inflammation caused by the present of sphingosine-1-phosphate in the emulsion.

While the preferred embodiments of the present invention have been illustrated in detail, it should be apparent that modifications and adaptations to those embodiments may occur to one skilled in the art without departing from the scope of the present invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the following description, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof

What is claimed is:

1. A method for treating sickle cell disease, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

2. The method of claim 1, wherein the pharmaceutical composition is a nanoemulsion.

3. The method of claim 1 or 2, wherein the fluorocarbon comprises perfluorobutane, perfluoropentane, perfluorohexane, perfluoroheptane, perfluorooctane, or a mixture of two of more thereof

4. The method of claim 3, wherein the fluorocarbon comprises perfluoropentane.

5. The method of any of claims 1-4, wherein the fluorocarbon accounts for a weight percent in the nanoemulsion from about 1% to about 50%.

6. The method of claim 5, wherein the fluorocarbon accounts for a weight percent in the nanoemulsion from about 1% to about 10%.

7. The method of any of claims 1-6, wherein the pharmaceutical composition comprises one or more phospholipids having carbon chains ranging from about 12 carbons to about 18 carbons in length.

8. The method of claim 7, wherein the phospholipids accounts for a weight percent in the pharmaceutical composition from about 0.10% to about 7.5%.

9. The method of any of claims 1-8, wherein the therapeutically effective dosage ranges from about 2% to about 4%.

10. The method of any of claims 1-8, wherein the therapeutically effective dosage ranges from about 0.5 to about 20 mg/Kg fluorocarbon.

11. The method of claim 10, wherein a dose of about 0.5 mg/Kg to about 5 mg/Kg is administered.

12. The method of claim 11, wherein a dose of about 1.5 mg/Kg to about 2.5 mg/Kg is administered.

13. The method of claim 11, wherein a dose of about 2.0 mg/Kg is administered.

14. The method of any of claims 11-13, wherein the dose is repeated from about 90 min. to about 120 min. apart for 2, 3, 4, 5 or 6 times.

15. The method of claim 14, wherein the dose is repeated from about 90 min. to about 120 min. apart for 4 times.

16. A method to treat a lung condition, comprising administering to a subject in need thereof a pharmaceutical composition comprising a therapeutically effective dosage of a fluorocarbon having a boiling point between about −4° C. and about +100° C., and a pharmaceutically acceptable carrier or excipient.

17. The method of claim 16, wherein the pharmaceutical composition is a nanoemulsion.

18. The method of claim 16 or 17, wherein the fluorocarbon comprises perfluorobutane, perfluoropentane, perfluorohexane, perfluoroheptane, perfluorooctane, or a mixture of two of more thereof

19. The method of claim 18, wherein the fluorocarbon comprises perfluoropentane.

20. The method of any of claims 16-19, wherein the fluorocarbon accounts for a weight percent in the nanoemulsion from about 1% to about 50%.

21. The method of claim 20, wherein the fluorocarbon accounts for a weight percent in the nanoemulsion from about 1% to about 10%.

22. The method of any of claims 16-21, wherein the pharmaceutical composition comprises one or more phospholipids having carbon chains ranging from about 12 carbons to about 18 carbons in length.

23. The method of claim 22, wherein the phospholipids accounts for a weight percent in the pharmaceutical composition from about 0.10% to about 7.5%.

24. The method of any of claims 16-23, wherein the therapeutically effective dosage ranges from about 2% to about 4%.

25. The method of any of claims 16-23, wherein the therapeutically effective dosage ranges from about 0.5 to about 20 mg/kg fluorocarbon.

26. A pharmaceutical composition comprising a dosage of a fluorocarbon having a boiling point between about −4° C. and about +100° C. therapeutically effective to treat sickle cell disease, or a related disease or disorder thereof, in a mammal, including a human, and a pharmaceutically acceptable carrier or excipient.

27. The pharmaceutical composition of claim 26, wherein the pharmaceutical composition is a nanoemulsion.

28. The pharmaceutical composition of claim 26 or 27, wherein the fluorocarbon comprises perfluorobutane, perfluoropentane, perfluorohexane, perfluoroheptane, perfluorooctane, or a mixture of two of more thereof

29. The pharmaceutical composition of claim 28, wherein the fluorocarbon comprises perfluoropentane.

30. The pharmaceutical composition of any of claims 26-29, wherein the fluorocarbon accounts for a weight percent in the nanoemulsion from about 1% to about 50%.

31. The pharmaceutical composition of claim 30, wherein the fluorocarbon accounts for a weight percent in the nanoemulsion from about 1% to about 10%.

32. The pharmaceutical composition of any of claims 26-31, wherein the pharmaceutical composition comprises one or more phospholipids having carbon chains ranging from about 12 carbons to about 18 carbons in length.

33. The pharmaceutical composition of claim 32, wherein the phospholipids accounts for a weight percent in the pharmaceutical composition from about 0.10% to about 7.5%.

34. A unit dosage form of a pharmaceutical composition in the form of a nanoemulsion comprising a dosage of a fluorocarbon having a boiling point between about −4° C. and about +100° C. therapeutically effective to treat sickle cell disease, or a related disease or disorder thereof, in a mammal, including a human, and a pharmaceutically acceptable carrier or excipient.

35. The unit dosage form of claim 34, wherein the fluorocarbon is selected from perfluorobutane, perfluoropentane, perfluorohexane, perfluoroheptane, perfluorooctane, or a mixture of two of more thereof

36. The unit dosage form of claim 35, wherein the fluorocarbon is perfluoropentane.

37. The unit dosage form of any of claims 34-36, wherein the fluorocarbon accounts for a weight percent in the nanoemulsion from about 1% to about 50%.

38. The unit dosage form of claim 37, wherein the fluorocarbon accounts for a weight percent in the nanoemulsion from about 1% to about 10%.

39. The unit dosage form of any of claims 34-38, wherein the nanoemulsion comprises one or more phospholipids having carbon chains ranging from about 12 carbons to about 18 carbons in length.

40. The unit dosage form of claim 39, wherein the phospholipids accounts for a weight percent in the nanoemulsion from about 0.10% to about 7.5%.

41. The unit dosage form of any of claims 34-40, comprising about 2% to about 4% of the fluorocarbon.

42. The unit dosage form of claim 41, comprising about 7 mg to about 140 mg of the fluorocarbon.
